(19) United States
(12) Patent Application Publication
(10) Pub. No.: US 2014/0212453 A1
(43) Pub. Date: Jul. 31, 2014

(54) SNACK SYSTEM, INCLUDING INTERIOR FILING OF INGREDIENT, OUTER COATING OF INGREDIENT, ADDITION OF INGREDIENT, AND FORMATION OF SNACK ROLL

(71) Applicant: Alice Chang, South Pasadena, CA (US)

(72) Inventor: Alice Chang, South Pasadena, CA (US)

(21) Appl. No.: 14/090,985

(22) Filed: Nov. 26, 2013

Related U.S. Application Data
(60) Provisional application No. 61/757,227, filed on Jan. 28, 2013.

(51) Int. Cl. A23L 1/00; A61K 31/49; A61K 36/00

(52) U.S. Cl. CPC A23L 1/0067; A61K 36/00; A61K 31/49
USPC 424/195.18; 426/582; 426/660; 426/631; 426/565; 426/656; 426/573; 426/579; 426/3; 426/549; 426/556; 426/659; 426/103; 426/94; 426/101; 426/89; 426/93; 426/583; 426/92; 426/72; 426/73; 426/74; 426/61; 426/62; 426/5; 514/305

(57) ABSTRACT

A novel snack system is created in various form, variety, and with various ingredient. The snack system is created being a snack, with at least one ingredient, and at least one texture. The category of snack includes cheese, powdery candy, soft candy, creamy candy, gummy candy, hard candy, liquid candy, fudge candy, chocolate, ice cream, ice milk, sherbet, gelato, yogurt, sorbet, tofu, jelly, pudding, chewing gum, roll, pie, biscuit, cookie, donut, pastry, cake, pancake, crepe, waffle, bread, tortilla, and taco. The snack can be served at room temperature, cold from refrigerator, warm, hot, and reheated. The snack is in form selected from group consisting of interior filing of ingredient, outer coating of ingredient, and formation of snack roll. The snack is also created with addition of various ingredients. The snack is created with at least one ingredient selected from group consisting of flavoring ingredient, nutritional ingredient, health ingredient, and other ingredient.

### CROSS-REFERENCE TO RELATED APPLICATION

The present application is related to and claims priority from prior provisional application No. 61/757,227, filed Jan. 28, 2013 and another prior provisional application No. 61/753,519, filed Jan. 18, 2013 which applications are incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of comestibles, and more specifically relates to a novel snack system, including interior filing of ingredient, outer coating of ingredient, addition of ingredient, and formation of snack roll.

2. Description of the Related Art

Currently on the market, snacks are very limited in their form and variety. There are limited creations of snacks with interior filing of ingredients, outer coating of ingredients, addition of ingredients, and formation of a snack roll.

Various attempts have been made to solve the above-mentioned problems such as those found in U.S. Pat. Nos. 4,925,683, 5,783,239, 6,911,226, 2010/0166933, 2011/0014320, 2012/0244258, 2012/0251678, 3864071, 4367243, 4411920, 6010731, 2003/0096039, 2003/0138539, 2009/0245698. This prior art is representative of comestibles relating to food. None of the above inventions and patents, taken either singly or in combination, is seen to describe the invention as claimed.

Ideally, the snack system, including interior filing of ingredient, outer coating of ingredient, addition of ingredient, and formation of snack roll, should provide consumers a wide selection of snack in various form, variety, and with various ingredient, yet manufactured at a modest expense.

### BRIEF SUMMARY OF THE INVENTION

In view of the foregoing disadvantages inherent in the known confectionery related comestibles art, the present invention provides a novel snack system. The general purpose of the present invention, which will be described subsequently in greater detail is to provide a snack system, including interior filing of ingredient, outer coating of ingredient, addition of ingredient, and formation of snack roll. The invention is to provide people who shop for snack the option of getting snack in various form, variety, and with various ingredient.

The snack system comprises snack, with at least one ingredient, and at least one texture. The category of snack is selected from the group consisting of cheese, powdery candy, soft candy, creamy candy, gummy candy, hard candy, liquid candy, fudge candy, chocolate, ice cream, ice milk, sherbet, gelato, yogurt, sorbet, tofu, jelly, pudding, chewing gum, roll, pie, biscuit, cookie, donut, pastry, cake, pancake, crepe, waffle, bread, tortilla, and taco. The snack is selected from the group consisting of caffeinated, and non caffeinated. The snack is selected from the group consisting of diet, and non diet. The snack can be uncooked. The snack can be cooked by method selected from the group consisting of deep frying, sun drying, roasting, baking, boiling, steaming, grilling, and frying. The snack is served in manner selected from the group consisting of room temperature, cold from refrigerator, warm, hot, and reheated. The snack comprising sugar can quickly replenish energy levels for the consumer. At least one texture is selected from the group consisting of powdery, soft, creamy, chewy, crunchy, crispy, jelly, gel, wax, liquid, oil, hard, semi solid, solid, and brittle.

The present invention holds significant improvements and serves as a novel snack system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### DETAILED DESCRIPTION

As discussed above, embodiments of the present invention relate to a comestible and more particularly to a novel snack system, including interior filing of ingredient, outer coating of ingredient, addition of ingredient, and formation of snack roll.

As mentioned earlier, the snack system comprises snack, with at least one ingredient, and at least one texture. The category of snack is selected from the group consisting of cheese, powdery candy, soft candy, creamy candy, gummy candy, hard candy, liquid candy, fudge candy, chocolate, ice cream, ice milk, sherbet, gelato, yogurt, sorbet, tofu, jelly, pudding, chewing gum, roll, pie, biscuit, cookie, donut, pastry, cake, pancake, crepe, waffle, bread, tortilla, and taco. The snack is selected from group consisting of caffeinated, and non caffeinated. The snack is selected from the group consisting of diet, and non diet. The snack can be uncooked. The snack can be cooked by method selected from the group consisting of deep frying, sun drying, roasting, baking, boiling, steaming, grilling, and frying. The snack is served in manner selected from the group consisting of room temperature, cold from refrigerator, warm, hot, and reheated. The snack comprising sugar can quickly replenish energy levels for the consumer. At least one texture is selected from the group consisting of powdery, soft, creamy, chewy, crunchy, crispy, jelly, gel, wax, liquid, oil, hard, semi solid, solid, and brittle.

The snack is created in form selected from the group consisting of having filing of ingredient, and without filing of ingredient. The filing of ingredient comprises at least one filing of at least one ingredient. The filing of ingredient is in form selected from the group consisting of even composition, uneven composition, horizontal layers of varying composition, vertical layers of varying composition, diagonal layers of varying composition, top layer, bottom layer, side layer, and back layer.

The snack is created in form selected from the group consisting of having coating of ingredient, and without coating of ingredient. The coating of ingredient comprises at least one coating of at least one ingredient. The coating of ingredient is in form selected from the group consisting of even composition, uneven composition, single layer, multiple layers, and topping.

The snack is created with at least one ingredient selected from the group consisting of at least one flavoring ingredient, at least one nutritional ingredient, at least one health ingredient, and at least one other ingredient.

The snack is created in form as formation of snack roll by method selected from the group consisting of placing at least one ingredient on top of dough before rolling; placing at least one ingredient inside roll after rolling; rolling dough from side to side; sealing off one end of roll; sealing off both ends of roll; leaving both ends of roll unsealed; folding over dough into half size, and sealing off edges of dough; shaping one dough into specific shape and size, and placing said at least one ingredient on top of dough; shaping two dough into specific shape and size, placing at least one ingredient on top of first dough, placing second dough on top of first, and sealing off edges of dough; and cooking by method selected from group consisting of deep frying, baking, steaming, roasting, grilling, and frying.

The dough is a thick, malleable, sometimes elastic, paste made by method selected from the group consisting of mixing flour with a small amount of water; grounding of substance selected from the group consisting of root vegetable, cereal grain, nut, seed, and bean; adding of substance selected from the group consisting of yeast, salt, mammal milk, plant milk, water, cheese, sugar, syrup, honey, vegetable shortening, plant oil, vegetable oil, vegetable fat, plant wax, animal wax, beeswax, carnauba wax, butter, margarine, animal fat, suet, lard, beaten egg, egg white, leavening agent, and at least one ingredient; rolling into a thin layer; rolling into a thick layer; and rolling into specific shape and size.

The snack roll having variety selected from the group consisting of pizza roll, bread roll, crepe roll, pancake roll, waffle roll, tortilla roll, taco roll, egg roll, vegetable roll, potato roll, taro roll, rice roll, oat roll, cereal roll, nut roll, seed roll, bean roll, biscuit roll, cookie roll, donut roll, pastry roll, and cake roll.

The snack is created as cheese by method selected from the group consisting of using milk, using whole milk, using low fat milk, using non fat milk, using buttermilk, using cream, separating milk into solid curds and liquid whey, using curds, using whey, adding acid, adding rennet, adding starter bacteria, adding thermophilic starter bacteria, adding swiss starter culture, adding salt, heating to temperature in the range of 35 to 55 degrees Celsius (95 to 131 degrees Fahrenheit), healing, cutting, stretching, cheddaring, draining, washing, pressing, applying pressure, adding bacteria, adding molds, and aging. The cheese created with a greater proportion of acid to rennet results in softer, smaller, and fresher varieties. The cheese created with a greater proportion of rennet to acid results in harder, larger, and longer-aged varieties. The milk is selected from the group consisting of mammal milk and plant milk. The cheese can be prepared as plain, and with at least one ingredient.

The snack is created as candy by method selected from the group consisting of dissolving sugar in water to form syrup, dissolving sugar in milk to form syrup, using syrup, using honey, adding at least one ingredient, and boiling at temperature from about 110 to 170 degrees Celsius (230 to 338 degrees Fahrenheit) until desired concentration is reached. Boiling at lower temperature creates softer candy. Boiling at higher temperature creates hard and brittle candy.

The snack is created as chocolate by method selected from the group consisting of addition of cocoa solids and cocoa butter at varying proportions, addition of mammal milk, addition of plant milk, and addition of at least one ingredient. The cocoa solids and cocoa butter are processed from chocolate liquor. The chocolate liquor is pure chocolate in fluid form liquefied from cocoa mass. The cocoa mass is ground nibs resulting from grounding of roasted riped unshelled cocoa beans after fermentation.

The snack is created as ice cream by method of heating mixture of chosen ingredients to 85 degrees Celsius (185 degrees Fahrenheit) for a pasteurization process, and slowly stifling mixture of chosen ingredients while cooling in order to incorporate air. The chosen ingredients are selected from the group consisting of mammal milk, plant milk, cream, yogurt, sweetener, at least 10 percent milk fat, and at least one ingredient.

The snack is created as ice milk by method of heating mixture of chosen ingredients to 85 degrees Celsius (185 degrees Fahrenheit) for a pasteurization process, and slowly stirring mixture of chosen ingredients while cooling in order to incorporate air. The chosen ingredients are selected from the group consisting of mammal milk, plant milk, cream, yogurt, sweetener, milkfat between 2 and 10 percent, and at least one ingredient.

The snack is created as sherbet by method of heating mixture of chosen ingredients to 85 degrees Celsius (185 degrees Fahrenheit) for a pasteurization process, and slowly stifling mixture of chosen ingredients while cooling in order to incorporate air. The chosen ingredients are selected from the group consisting of mammal milk, plant milk, cream, yogurt, sweetener, milkfat between 1 and 2 percent, and said at least one ingredient.

The snack is created as gelato by method of heating mixture of chosen ingredients to 85 degrees Celsius (185 degrees Fahrenheit) for a pasteurization process, and slowly stifling mixture of chosen ingredients while cooling in order to incorporate a small amount of air. The chosen ingredients are selected from the group consisting of mammal milk, plant milk, cream, yogurt, sweetener, at least 3.5 percent butter fat, and at least one ingredient.

The snack is created as yogurt by method selected from the group consisting of heating milk to about 80 degrees Celsius (176 degrees Fahrenheit) for a pasteurization process to kill any undesirable bacteria and to denature milk proteins so that they set together rather than form curds, cooling milk to about 45 degrees Celsius (112 degrees Fahrenheit), adding bacterial culture, and allowing 4 to 7 hours for fermentation; said milk is selected from group consisting of mammal milk and plant milk; and said yogurt can be prepared as plain, and with at least one ingredient.

The snack is created as sorbet by method selected from the group consisting of freezing fruit juice, freezing fruit juice with added wine, freezing fruit juice with added liquor, adding at least one ingredient before freezing, and crushing frozen ice.

The snack is created as tofu by method selected from the group consisting of soaking, grinding, boiling and straining dried soybeans to create soymilk; soaking, grinding, boiling and straining fresh soybeans to create soymilk; adding said at least one ingredient; coagulating soymilk with aid of coagulants including salts, acids, and enzymes; and pressing the resulting curds.

The snack is created as jelly by method selected from the group consisting of boiling gelatin with water, adding at least one ingredient, and allowing to cool and congeal below 15 degrees Celsius (59 degrees Fahrenheit).

The snack is created as pudding by method selected from the group consisting of mixing chosen ingredients, and cooking by method selected from the group consisting of deep frying, roasting, baking, steaming, boiling, grilling, and frying. The chosen ingredients are selected from the group consisting of flour, root vegetable, cereal grain, nut, seed, bean, mammal milk, plant milk, cheese, sugar, syrup, honey, plant oil, vegetable oil, butter, margarine, vegetable fat, animal fat, plant wax, animal wax, beeswax, carnauba wax, suet, lard, beaten egg, egg white, and said at least one ingredient.

The snack is created as chewing gum by mixing substance selected from the group consisting of chicle, resin, latex, natural gum, styrene-butadiene rubber, isobutylene, isoprene copolymer, paraffin wax, petroleum wax, xylitol, sorbitol, artificial sweetener, sugar, fluoride, triclosan, p-chlorbenzyl-4-methylbenzylpiperazine, calcium lactate, propolis, plant wax, animal wax, beeswax, carnauba wax, and at least one ingredient.

The snack is in varying size. The varying size is selected from the group consisting of bite size, snack bar, and large size for multiple servings.

The snack is in varying shape and pattern. The varying shape and pattern is selected from the group consisting of geometrical and non geometrical shape and pattern in varying sizes, including sphere, saucer, disc, tube, cube, cylinder, cone, dome, prism, antiprism, triangle, hexagon, octagon, rectangle, square, circle, oval, ellipse, curve, arrow, cross, wave, stripe, paisley, heart, ring, knot, spike, braid, twist, star, snowflake, spiral, and so on. The varying shape and pattern is at location selected from the group consisting of filing, coating, and topping.

At least one filing is in form selected from the group consisting of extract, juice, sauce, cream, liquid, oil, gel, jelly, wax, paste, powder, thin layer, small particles, small pieces, semi solid, and solid. At least one filing in form of liquid includes water, juice, oil, and alcohol.

At least one filing in form of liquid is selected from the group consisting of tea drink, coffee drink, milk drink, mammal milk, plant milk, milk shake, yogurt drink, malted drink, roasted cereal grain beverage, roasted nut and seed beverage, roasted bean beverage, distilled water, mineral water, sports drink, fruit juice, vegetable juice, fruit drink, vegetable drink, fruit skin drink, vegetable skin drink, plant drink, soft drink, alcoholic drink, beer, wine, liquor, cocktail, soup drink, plant oil, vegetable oil, fish oil, fish liver oil, and at least one ingredient.

At least one filing in form of extract, sauce, cream, gel, jelly, wax, paste, powder, thin layer, small pieces, semi solid, and solid is selected from the group consisting of artificial sweetener, sugar, syrup, honey, chocolate, cream, sour cream, ice cream, yogurt, cheese, cream cheese, cottage cheese, mozzarella, mascarpone, ricotta, butter, margarine, royal jelly, caramel, butterscotch, peanut butter, cookie, biscuit, cake, jelly, malt, fruit, fruit skin, vegetable, vegetable skin, nut, seed, cereal grain, bean, plant, herb, egg, meat, seafood, vegetable shortening, vegetable fat, animal fat, plant wax, animal wax, beeswax, carnauba wax, and at least one ingredient.

At least one coating is selected from the group consisting of having an outer layer of casing selected from the group consisting of intestine, and cellulose; comprising at least one coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, chocolate, fudge, and at least one ingredient.

At least one flavoring ingredient is selected from the group consisting of natural flavor, artificial flavor, fruit flavor, plant flavor, plant oil flavor, herb flavor, tea flavor, root flavor, flower flavor, flower bud flavor, pollen flavor, leaf flavor, stem flavor, stem shoot flavor, bulb flavor, nut flavor, seed flavor, bean flavor, cereal grain flavor, fruit skin flavor, vegetable skin flavor, vegetable flavor, vegetable oil flavor, vegetable fat flavor, meat flavor, seafood flavor, and other flavor. The at least one flavoring ingredient is in form selected from the group consisting of skin, skinned, unskinned, unshelled, cut, uncut, extract, juice, sauce, liquid, oil, gel, jelly, wax, cream, paste, powder, thin layer, small particles, and small pieces. The at least one flavoring ingredient can be uncooked, and cooked by method selected from the group consisting of deep frying, sun drying, roasting, baking, steaming, boiling, grilling, and frying.

For at least one flavoring ingredient, other flavor is selected from the group consisting of artificial sweetener, salt, vinegar, sugar, syrup, honey, chocolate, coffee, tea, cream, sour cream, milk, mammal milk, plant milk, ice cream, yogurt, cheese, cream cheese, cottage cheese, mozzarella, mascarpone, ricotta, butter, margarine, royal jelly, caramel, butterscotch, peanut butter, cookie, biscuit, cake, jelly, pudding, custard, bread, pita, tortilla, pancake, crepe, candy, marshmallow, fish oil, fish liver oil, vegetable shortening, plant wax, animal wax, beeswax, carnauba wax, tonic water, mineral water, glycerol, egg, and malt.

For at least one flavoring ingredient, fruit flavor and fruit skin flavor are selected from the group consisting of apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, marula, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortińo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, and zig zag vine.

For at least one flavoring ingredient, vegetable flavor, vegetable skin flavor, vegetable oil flavor, and vegetable fat flavor are selected from the group consisting of root vegetable, fruit vegetable, flower bud, flower, stem shoot, stem, leaf, bulb, cereal grain, nut, seed, bean, sea vegetable, and other types of vegetable, including aloe vera, jojoba, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, horseradish, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turmeric, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, chrysanthemum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, layer, mozuku, nori, ogonori, sea grape, sea lettuce, sea weed, wakame, truffle, mushroom, and bean sprout.

For at least one flavoring ingredient, plant flavor, plant oil flavor, herb flavor, tea flavor, root flavor, flower flavor, flower bud flavor, pollen flavor, leaf flavor, stem flavor, stem shoot flavor, bulb flavor, nut flavor, seed flavor, bean flavor, and cereal grain flavor are selected from the group consisting of leaf, flower, flower bud, pollen, bulb, root, stem, stem shoot, and herb, including hop, menthol, echinacea, elder, horehound, mallow, sage, thyme, cowslip, burnet, yarrow, marsh mallow, lady's mantle, speedwell, plantain, linden flowers, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, turmeric, mustard, curry, wasabi, mountain mint, peppermint, spearmint, wintergreen, cinnamon, licorice, rose, jasmine, chamomile, chrysanthemum, lavender, acacia, safflower, vanilla, asafoetida, balsam, bay, buchu, birch, camphor, cannabis, caraway, calamus, cistus, citronella, clary sage, costus, cubeb, cypress, cypriol, davana, elecampane, fenugreek, fir, frankincense, galbanum, geranium, goldenrod, henna, helichrysum, hickory, juniper, ledum, litseacubeba, marjoram, melaleuca, mountain savory, mugwort, myrrh, myrtle, neroli, nutmeg, orris, palosanto, petitgrain, pine, ravensara, red cedar, rosewood, sage, sassafras, schisandra, spikenard, spruce, tea tree, tsuga, valerian, yarrow, ylangylang, zedoary, benzoin, copaiba, manuka, bergamot, patchouli, lilium, ambergris, gardenia, oakmoss, labdanum, coumarin, iris, water lily, ivy, amber, carnation, vetiver, cardamom, freesia, cyclamen, muguet, gardenia, tuberose, tonka bean, agarwood, cedarwood, sandalwood, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortińo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, layer, mozuku, nori, ogonori, sea grape, sea lettuce, sea weed, wakame, truffle, mushroom, bean sprout, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, jojoba, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, file powder, fingerroot, galangal, galingale, ganoderma, golpar, grains of paradise, grains of selim, houttuynia cordata, huacatay, indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, and woodruff.

For at least one flavoring ingredient, vegetable oil flavor is selected from the group consisting of corn oil, canola oil, peanut oil, olive oil, coconut oil, soybean oil, sesame oil, sunflower oil, flaxseed oil, grape seed oil, pumpkin seed oil, safflower oil, argan oil, rice bran oil, palm oil, palm kernel oil, almond oil, avocado oil, cottonseed oil, hemp oil, mustard oil, macadamia oil, groundnut oil, teaseed oil, walnut oil, cashew oil, castor oil, colza oil, hazelnut oil, linseed oil, manila oil, mongongo nut oil, mustard oil, pecan oil, perilla oil, pine nut oil, pistachio oil, poppyseed oil, rapeseed oil, jojoba oil, and watermelon seed oil.

For at least one flavoring ingredient, meat flavor is selected from the group consisting of chicken, duck, goose, turkey, quail, rabbit, sheep, goat, cow, pig, deer, bison, buffalo, crocodile, frog, turtle, snail, worm, bacon, sausage, animal tissue, animal organ, animal tongue, animal tail, animal feet, animal liver, animal stomach, animal intestine, animal brain, animal heart, animal skin, animal tendon, animal fat, animal bone marrow, and animal blood.

For at least one flavoring ingredient, seafood flavor is selected from the group consisting of squid, cuttle fish, shrimp, crab, crayfish, octopus, clam, oyster, scallop, lobster, abalone, conch, jellyfish, prawn, krill, mussel, sea cucumber, sea urchin, eel, snail, shrimp roe, crab roe, lobster roe, fish roe, and fish.

At least one nutritional ingredient comprises essential nutrient selected from the group consisting of vitamin, dietary mineral, amino acid, and fatty acid. The vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K. The dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium. The amino acid includes histidine, isoleucine, leucine, lysine, methionine, cysteine, phenylalanine, tyrosine, threonine, tryptophan, and valine. The fatty acid includes alpha-linoleic acid, and linoleic acid. The at least one nutritional ingredient is in form selected from the group consisting of extract, juice, sauce, liquid, oil, gel, jelly, wax, cream, paste, powder, thin layer, small particles, and small pieces.

At least one health ingredient comprises medication and other health ingredient. The medication is mixed into the snack for ease of consumption. The other health ingredient is selected from the group consisting of electrolyte, lactase, yeast, propolis, and quinine. The at least one health ingredient is in form selected from the group consisting of extract, juice, sauce, liquid, oil, gel, jelly, wax, cream, paste, powder, thin layer, small particles, and small pieces.

At least one other ingredient comprises food additive selected from the group consisting of acid, acidity regulator, alcohol, anticaking agent, antifoaming agent, antioxidant, bulking agent, coagulation agent, food coloring, color retention agent, emulsifier, flavor enhancer, flour treatment agent, gelling agent, glazing agent, humectant, leavening agent, tracer gas, preservative, stabilizer, sweetener, tenderizer, and thickener.

At least one ingredient further comprises at least one binding agent which is an edible binder and is selected from the group consisting of protein component and starch component. The protein component is selected from the group consisting of gelatin, beaten egg, and egg white. The starch component is selected from the group consisting of vegetable gum, vegetable starch, and vegetable flour. The vegetable gum is selected from the group consisting of agar, alginic acid, sodium alginate, carrageenan, gum arabic, gum ghatti, gum tragacanth, karaya gum, guar gum, locust bean gum, beta glucan, chicle gum, dammar gum, glucomannan gum, mastic gum, psyllium seed husk, spruce gum, tara gum, gellan gum, and xanthan gum. The vegetable starch and vegetable flour are selected from the group consisting of root vegetable, cereal grain, nut, seed, bean, pectin, sago, and tapioca. The at least one binding agent can be cooked by method selected from the group consisting of mixing with water, boiling, and steaming. The gelatin from fish and animal congeals below 15 degrees Celsius (59 degrees Fahrenheit).

The snack with at least one binding agent comprising gelatin can be refrigerated to serve cold but not reheated because gelatin from fish and animal congeals below 15 degrees Celsius (59 degrees Fahrenheit). The snack with at least one binding agent selected from the group consisting of beaten egg, egg white, and starch component can be served at room temperature, refrigerated to serve cold, and reheated to serve hot.

The snack having packaging selected from the group consisting of individual packaging and multiple packaging. The packaging is in air tight container. The packaging has material selected from group consisting of paper, plastic, metal, and glass.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent set forth in the appended claims:

1. A snack system comprising:
a snack, with
at least one ingredient, and
at least one texture;
wherein said snack including category is selected from group consisting of cheese, powdery candy, soft candy, creamy candy, gummy candy, hard candy, liquid candy, fudge candy, chocolate, ice cream, ice milk, sherbet, gelato, yogurt, sorbet, tofu, jelly, pudding, chewing gum, roll, pie, biscuit, cookie, donut, pastry, cake, pancake, crepe, waffle, bread, tortilla, and taco;
wherein said snack is selected from group consisting of caffeinated, and non caffeinated;
wherein said snack is selected from group consisting of diet, and non diet;
wherein said snack can be uncooked;
wherein said snack can be cooked by method selected from group consisting of deep frying, sun drying, roasting, baking, boiling, steaming, grilling, and frying;
wherein said snack is served in manner selected from group consisting of room temperature, cold from refrigerator, warm, hot, and reheated;
wherein said snack comprising sugar can quickly replenish energy levels for consumer; and
wherein said at least one texture is selected from group consisting of powdery, soft, creamy, chewy, crunchy, crispy, jelly, gel, wax, liquid, oil, hard, semi solid, solid, and brittle.

2. The snack system of claim 1 wherein said snack is created in form selected from group consisting of having filing of ingredient, and without filing of ingredient; said filing of ingredient comprises at least one filing of said at least one ingredient; and said filing of ingredient is in form selected from group consisting of even composition, uneven composition, horizontal layers of varying composition, vertical layers of varying composition, diagonal layers of varying composition, top layer, bottom layer, side layer, and back layer.

3. The snack system of claim 1 wherein said snack is created in form selected from group consisting of having coating of ingredient, and without coating of ingredient; said coating of ingredient comprises at least one coating of said at least one ingredient; and said coating of ingredient is in form selected from group consisting of even composition, uneven composition, single layer, multiple layers, and topping.

4. The snack system of claim 1 wherein said snack is created with said at least one ingredient selected from group consisting of at least one flavoring ingredient, at least one nutritional ingredient, at least one health ingredient, and at least one other ingredient.

5. The snack system of claim 1 wherein said snack is created in form as formation of snack roll by method selected from group consisting of placing said at least one ingredient on top of dough before rolling; placing said at least one ingredient inside roll after rolling; rolling dough from side to side; sealing off one end of roll; sealing off both ends of roll; leaving both ends of roll unsealed; folding over dough into half size, and sealing off edges of dough; shaping one dough into specific shape and size, and placing said at least one ingredient on top of dough; shaping two dough into specific shape and size, placing said at least one ingredient on top of first dough, placing second dough on top of first, and sealing off edges of dough; and cooking by method selected from group consisting of deep frying, baking, steaming, roasting, grilling, and frying.

6. The snack system of claim 5 wherein said dough is a thick, malleable, sometimes elastic, paste made by method selected from group consisting of mixing flour with a small amount of water; grounding of substance selected from group consisting of root vegetable, cereal grain, nut, seed, and bean; adding of substance selected from group consisting of yeast, salt, mammal milk, plant milk, water, cheese, sugar, syrup, honey, vegetable shortening, plant oil, vegetable oil, vegetable fat, plant wax, animal wax, beeswax, carnauba wax, butter, margarine, animal fat, suet, lard, beaten egg, egg white, leavening agent, and said at least one ingredient; rolling into a thin layer; rolling into a thick layer; and rolling into specific shape and size.

7. The snack system of claim 5 wherein said snack roll having variety selected from group consisting of pizza roll, bread roll, crepe roll, pancake roll, waffle roll, tortilla roll, taco roll, egg roll, vegetable roll, potato roll, taro roll, rice roll, oat roll, cereal roll, nut roll, seed roll, bean roll, biscuit roll, cookie roll, donut roll, pastry roll, and cake roll.

8. The snack system of claim 1 wherein said snack is created as cheese by method selected from group consisting of using milk, using whole milk, using low fat milk, using non fat milk, using buttermilk, using cream, separating milk into solid curds and liquid whey, using curds, using whey, adding acid, adding rennet, adding starter bacteria, adding thermophilic starter bacteria, adding swiss starter culture, adding salt, heating to temperature in the range of 35 to 55 degrees Celsius, healing, cutting, stretching, cheddaring, draining, washing, pressing, applying pressure, adding bacteria, adding molds, and aging; said cheese created with a greater proportion of acid to rennet results in softer, smaller, and fresher varieties; said cheese created with a greater proportion of rennet to acid results in harder, larger, and longer-aged varieties; said milk is selected from group consisting of mammal milk and plant milk; and said cheese can be prepared as plain, and with said at least one ingredient.

9. The snack system of claim 1 wherein said snack is created as candy by method selected from group consisting of dissolving sugar in water to form syrup, dissolving sugar in milk to form syrup, using syrup, using honey, adding said at least one ingredient, and boiling at temperature from about 110 to 170 degrees Celsius until desired concentration is reached; said boiling at lower temperature creates softer candy; and said boiling at higher temperature creates hard and brittle candy.

10. The snack system of claim 1 wherein said snack is created as chocolate by method selected from group consisting of addition of cocoa solids and cocoa butter at varying proportions, addition of mammal milk, addition of plant milk, and addition of said at least one ingredient; said cocoa solids and cocoa butter are processed from chocolate liquor; said chocolate liquor is pure chocolate in fluid form liquefied from cocoa mass; and said cocoa mass is ground nibs resulting from grounding of roasted riped unshelled cocoa beans after fermentation.

11. The snack system of claim 1 wherein said snack is created as ice cream by method of heating mixture of chosen ingredients to 85 degrees Celsius for a pasteurization process, and slowly stirring mixture of chosen ingredients while cooling in order to incorporate air; said chosen ingredients are selected from group consisting of mammal milk, plant milk, cream, yogurt, sweetener, at least 10 percent milk fat, and said at least one ingredient.

12. The snack system of claim 1 wherein said snack is created as ice milk by method of heating mixture of chosen ingredients to 85 degrees Celsius for a pasteurization process, and slowly stirring mixture of chosen ingredients while cooling in order to incorporate air; said chosen ingredients are selected from group consisting of mammal milk, plant milk, cream, yogurt, sweetener, milkfat between 2 and 10 percent, and said at least one ingredient.

13. The snack system of claim 1 wherein said snack is created as sherbet by method of heating mixture of chosen ingredients to 85 degrees Celsius for a pasteurization process, and slowly stirring mixture of chosen ingredients while cooling in order to incorporate air; said chosen ingredients are selected from group consisting of mammal milk, plant milk, cream, yogurt, sweetener, milkfat between 1 and 2 percent, and said at least one ingredient.

14. The snack system of claim 1 wherein said snack is created as gelato by method of heating mixture of chosen ingredients to 85 degrees Celsius for a pasteurization process, and slowly stirring mixture of chosen ingredients while cooling in order to incorporate a small amount of air; said chosen ingredients are selected from group consisting of mammal milk, plant milk, cream, yogurt, sweetener, at least 3.5 percent butter fat, and said at least one ingredient.

15. The snack system of claim 1 wherein said snack is created as yogurt by method selected from group consisting of heating milk to about 80 degrees Celsius for a pasteurization process to kill any undesirable bacteria and to denature milk proteins so that they set together rather than form curds, cooling milk to about 45 degrees Celsius, adding bacterial culture, and allowing 4 to 7 hours for fermentation; said milk is selected from group consisting of mammal milk and plant milk; and said yogurt can be prepared as plain, and with said at least one ingredient.

16. The snack system of claim 1 wherein said snack is created as sorbet by method selected from group consisting of freezing fruit juice, freezing fruit juice with added wine, freezing fruit juice with added liquor, adding said at least one ingredient before freezing, and crushing frozen ice.

17. The snack system of claim 1 wherein said snack is created as tofu by method selected from group consisting of soaking, grinding, boiling and straining dried soybeans to create soymilk; soaking, grinding, boiling and straining fresh soybeans to create soymilk; adding said at least one ingredient; coagulating soymilk with aid of coagulants including salts, acids, and enzymes; and pressing the resulting curds.

18. The snack system of claim 1 wherein said snack is created as jelly by method selected from group consisting of boiling gelatin with water, adding said at least one ingredient, and allowing to cool and congeal below 15 degrees Celsius.

19. The snack system of claim 1 wherein said snack is created as pudding by method selected from group consisting of mixing chosen ingredients, and cooking by method selected from group consisting of deep frying, roasting, baking, steaming, boiling, grilling, and frying; and said chosen ingredients are selected from group consisting of flour, root vegetable, cereal grain, nut, seed, bean, mammal milk, plant milk, cheese, sugar, syrup, honey, plant oil, vegetable oil, butter, margarine, vegetable fat, animal fat, plant wax, animal wax, beeswax, carnauba wax, suet, lard, beaten egg, egg white, and said at least one ingredient.

20. The snack system of claim 1 wherein said snack is created as chewing gum by mixing substance selected from group consisting of chicle, resin, latex, natural gum, styrene-butadiene rubber, isobutylene, isoprene copolymer, paraffin wax, petroleum wax, xylitol, sorbitol, artificial sweetener, sugar, fluoride, triclosan, p-chlorbenzyl-4-methylbenzylpiperazine, calcium lactate, propolis, plant wax, animal wax, beeswax, carnauba wax, and said at least one ingredient.

21. The snack system of claim 1 wherein said snack is in varying size; and said varying size is selected from group consisting of bite size, snack bar, and large size for multiple servings.

22. The snack system of claim 1 wherein said snack is in varying shape and pattern; said varying shape and pattern is selected from group consisting of geometrical and non geometrical shape and pattern in varying sizes, including sphere, saucer, disc, tube, cube, cylinder, cone, dome, prism, antiprism, triangle, hexagon, octagon, rectangle, square, circle, oval, ellipse, curve, arrow, cross, wave, stripe, paisley, heart, ring, knot, spike, braid, twist, star, snowflake, and spiral; and said varying shape and pattern is at location selected from group consisting of filing, coating, and topping.

23. The snack system of claim 2 wherein said at least one filing is in form selected from group consisting of extract, juice, sauce, cream, liquid, oil, gel, jelly, wax, paste, powder, thin layer, small particles, small pieces, semi solid, and solid; and said at least one filing in form of liquid includes water, juice, oil, and alcohol.

24. The snack system of claim 2 wherein said at least one filing in form of liquid is selected from group consisting of tea drink, coffee drink, milk drink, mammal milk, plant milk, milk shake, yogurt drink, malted drink, roasted cereal grain beverage, roasted nut and seed beverage, roasted bean beverage, distilled water, mineral water, sports drink, fruit juice, vegetable juice, fruit drink, vegetable drink, fruit skin drink, vegetable skin drink, plant drink, soft drink, alcoholic drink, beer, wine, liquor, cocktail, soup drink, plant oil, vegetable oil, fish oil, fish liver oil, and said at least one ingredient.

25. The snack system of claim 2 wherein said at least one filing in form of extract, sauce, cream, gel, jelly, wax, paste, powder, thin layer, small pieces, semi solid, and solid is selected from group consisting of artificial sweetener, sugar, syrup, honey, chocolate, cream, sour cream, ice cream, yogurt, cheese, cream cheese, cottage cheese, mozzarella, mascarpone, ricotta, butter, margarine, royal jelly, caramel, butterscotch, peanut butter, cookie, biscuit, cake, jelly, malt, fruit, fruit skin, vegetable, vegetable skin, nut, seed, cereal grain, bean, plant, herb, egg, meat, seafood, vegetable shortening, vegetable fat, animal fat, plant wax, animal wax, beeswax, carnauba wax, and said at least one ingredient.

26. The snack system of claim 3 wherein said at least one coating is selected from group consisting of having an outer layer of casing selected from group consisting of intestine, and cellulose; and comprising said at least one coating selected from group consisting of bread crumbs, beer batter, egg batter, cornmeal batter, milk batter, yogurt batter, tea batter, coffee batter, malt batter, chocolate batter, honey batter, syrup batter, caramel batter, butterscotch batter, peanut butter batter, butter batter, margarine batter, cheese batter, cereal grain batter, nut batter, seed batter, bean batter, vegetable batter, plant batter, herb batter, fruit batter, wine batter, liquor batter, alcoholic batter, soft drink batter, soup batter, plant wax, animal wax, beeswax, carnauba wax, donut, crepe, pancake, pita, tortilla, bread, taco, biscuit, cookie, cake, jelly, royal jelly, cheese, cream cheese, cottage cheese, mozzarella, mascarpone, ricotta, butter, margarine, pudding, custard, candy, chocolate, fudge, and said at least one ingredient.

27. The snack system of claim 4 wherein said at least one flavoring ingredient is selected from group consisting of natural flavor, artificial flavor, fruit flavor, plant flavor, plant oil flavor, herb flavor, tea flavor, root flavor, flower flavor, flower bud flavor, pollen flavor, leaf flavor, stem flavor, stem shoot flavor, bulb flavor, nut flavor, seed flavor, bean flavor, cereal grain flavor, fruit skin flavor, vegetable skin flavor, vegetable flavor, vegetable oil flavor, vegetable fat flavor, meat flavor, seafood flavor, and other flavor; said at least one flavoring ingredient is in form selected from group consisting of skin, skinned, unskinned, unshelled, cut, uncut, extract, juice, sauce, liquid, oil, gel, jelly, wax, cream, paste, powder, thin layer, small particles, and small pieces; and said at least one flavoring ingredient can be uncooked, and cooked by method selected from group consisting of deep frying, sun drying, roasting, baking, steaming, boiling, grilling, and frying.

28. The snack system of claim 27 wherein said other flavor is selected from group consisting of artificial sweetener, salt, vinegar, sugar, syrup, honey, chocolate, coffee, tea, cream, sour cream, milk, mammal milk, plant milk, ice cream, yogurt, cheese, cream cheese, cottage cheese, mozzarella, mascarpone, ricotta, butter, margarine, royal jelly, caramel, butterscotch, peanut butter, cookie, biscuit, cake, jelly, pudding, custard, taco, bread, pita, tortilla, pancake, crepe, candy, marshmallow, fish oil, fish liver oil, vegetable shortening, plant wax, animal wax, beeswax, carnauba wax, tonic water, mineral water, glycerol, egg, and malt.

29. The snack system of claim 27 wherein said fruit flavor and fruit skin flavor are selected from group consisting of apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, marula, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, and zig zag vine.

30. The snack system of claim 27 wherein said vegetable flavor, vegetable skin flavor, vegetable oil flavor, and vegetable fat flavor are selected from group consisting of root vegetable, fruit vegetable, flower bud, flower, stem shoot, stem, leaf, bulb, cereal grain, nut, seed, bean, sea vegetable, and other types of vegetable, including aloe vera, jojoba, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, horseradish, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turmeric, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, chrysanthemum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, layer, mozuku, nori, ogonori, sea grape, sea lettuce, sea weed, wakame, truffle, mushroom, and bean sprout.

31. The snack system of claim 27 wherein said plant flavor, plant oil flavor, herb flavor, tea flavor, root flavor, flower flavor, flower bud flavor, pollen flavor, leaf flavor, stem flavor, stem shoot flavor, bulb flavor, nut flavor, seed flavor, bean flavor, and cereal grain flavor are selected from group consisting of leaf, flower, flower bud, pollen, bulb, root, stem, stem shoot, and herb, including hop, menthol, echinacea, elder, horehound, mallow, sage, thyme, cowslip, burnet, yarrow, marsh mallow, lady's mantle, speedwell, plantain, linden flowers, wild thyme, hyssop, eucalyptus, sea buckthorn, elder blossom, verbena, lemon balm, clove, coriander, cumin, basil, dill, rosemary, parsley, fennel, oregano, allspice, catnip, wormwood, musk, chili, horseradish, turmeric, mustard, curry, wasabi, mountain mint, peppermint, spearmint, wintergreen, cinnamon, licorice, rose, jasmine, chamomile, chrysanthemum, lavender, acacia, safflower, vanilla, asafoetida, balsam, bay, buchu, birch, camphor, cannabis, caraway, calamus, cistus, citronella, clary sage, costus, cubeb, cypress, cypriol, davana, elecampane, fenugreek, fir, frankincense, galbanum, geranium, goldenrod, henna, helichrysum, hickory, juniper, ledum, litseacubeba, marjoram, melaleuca, mountain savory, mugwort, myrrh, myrtle, neroli, nutmeg, orris, palosanto, petitgrain, pine, ravensara, red cedar, rosewood, sage, sassafras, schisandra, spikenard, spruce, tea tree, tsuga, valerian, yarrow, ylangylang, zedoary, benzoin, copaiba, manuka, bergamot, patchouli, lilium, ambergris, gardenia, oakmoss, labdanum, coumarin, iris, water lily, ivy, amber, carnation, vetiver, cardamom, freesia, cyclamen, muguet, gardenia, tuberose, tonka bean, agarwood, cedarwood, sandalwood, alfafa, apple, orange, lemon, lime, grapefruit, grape, guava, plum, peach, pear, blackberry, blueberry, strawberry, cranberry, raspberry, cherry, mango, pineapple, pomegranate, papaya, watermelon, rockmelon, hamimelon, melon, honeydew, coconut, durian, passion fruit, banana, fig, apricot, mandarin, blackcurrant, starfruit, kiwi, pomelo, lychee, loquat, persimmon, tangerine, mangosteen, noni, sugar apple, mulberry, elderberry, gooseberry, lingonberry, juniper berry, jackfruit, dragonfruit, abiu, acai, acerola, ackee, cherry orange, moringa, ambarella, araza, arhat, atemoya, atherton raspberry, babaco, bacupari, bacuri, bael, barbadine, beach plum, batuan, bearberry, bignay, bilberry, bilimbi, biriba, black apple, black cherry, black raspberry, black mulberry, black sapote, blood orange, bolwarra, boquila, blue tongue, breadfruit, broad leaf bramble, buddha's hand, buffaloberry, brush cherry, burdekin plum, bush tomato, button mangosteen, caimito, calabash tree, calamondin, calamansi, camucamu, canary melon, canistel, cape gooseberry, capuli cherry, carambola, cardon, carob, cashew apple, cassabanana, cattley guava, cawesh, cedar bay cherry, ceriman, cereus peruvianus, charichuelo, chayote, che, chempedak, chenet, cherimoya, cherry ballart, chokeberry, chokecherry, chupa chupa, citron, clementine, cloudberry, cluster fig, cocky apple, coco plum, cocona, cocoplum, coffee cherry, common apple berry, conkerberry, cornelian cherry, crowberry, cupuacu, currant, curry leaf apple, custard apple, elephant apple, key apple, damson plum, date, date plum, davidson's plum, dead man's finger, desert banana, desert fig, desert lime, dodder laurel, doubah, double coconut, duku, eastern may hawthorn, emu apple, emu berry, false mastic, feijoa, fibrous satinash, finger lime, florida strangler fig, gac, galia melon, gambooge, genip, giant granadilla, otaheite gooseberry, goumi, governor's plum, montessa granadilla, yellow granadilla, greengage, grumichama, guanabana, guarana, cattley guava, pineapple guava, purple guava, strawberry guava, guavaberry, hackberry, hairless rambutan, hardy kiwi, hawthorn, hog plum, honeysuckle, horned melon, huckleberry, huito, ice cream bean, llama, lllawarra plum, imbe, jabuticaba, jambul, jatoba, jelly palm, jocote, jujube, kabosu, kaffir lime, kahikatea, kakadu lime, kakadu plum, kapok, karkalla, karonda, kei apple, kepel fruit, keule, key lime, kitembilla, korlan, kumquat, kutjera, kwai muk, lady apple, lakoocha, langsat, lanzone, lapsi, lardizabala, lemon aspen, leucaena, lillypilly, little gooseberry tree, limequat, loganberry, longan, lucuma, ma praang, mabolo, madrono, malabar plum, mamey sapote, mamoncillo, mangaba, manoao, maqui, marang, manila, mayapple, malay apple, mammee apple, maypop, medlar, melinjo, melon pear, midyim, miracle fruit, mock strawberry, purple mombin, red mombin, yellow mombin, monstera, mora de castilla, morinda, morinda citrifolia, mortiflo, mountain pepper, mountain soursop, wild plum, mundu, muntries, muscadine, muskmelon, naartjie, nageia, nance, nannyberry, naranjilla, neem, nere, nonda plum, nungu, oil palm, oregan grape, oroblanco, otaheite gooseberry, palmyra palm, pawpaw, peachpalm, peanut butter fruit, pepino, pequi, peumo, phalsa, pigeon pea, pigeon plum, pigface, pindo palm, pineapple guava, pitomba, pois doux, pommecythere, pommerac, prickly pear, prune, pulasan, pummelo, pupunha, purple apple berry, purple granadilla, quandong, queensland ebony, quince, raisin tree, rambutan, red granadilla, red mulberry, rhubarb, riberry, rimu, rollinia, rose apple, rose hip, rose leaf bramble, rose myrtle, rowan, safou, sageretia, saguaro, salak, salal, salmonberry, sandpaper fig, santol, mamey, sapodilla, fibrous satinash, saskatoonberry, saw palmetto, sea buckthorn, serviceberry, shipova, small leaf tamarind, snow berry, soncoya, servicetree, soursop, southern crabapple, spanish lime, spanish tamarind, spiny monkey orange, star apple, carambola, strawberry guava, ugni, strawberry pear, surinam cherry, sweet apple berry, sweet ganadilla, sweet lemon, sweet orange, sweet pepper, sweetsop, sycamore fig, tamarillo, tamarind, monkey tamarind, velvet tamarind, tanjong, texas persimmon, thimbleberry, totara, toyon, wampee, water apple, wax apple, wax jambu, acerola, white aspen, white mulberry, white sapote, wild grape, wild orange, wild peach, wild plum, wild mangosteen, wineberry, wolfberry, wongi, wood apple, yangmei, yellow plum, yew, youngberry, zig zag vine, ahipa, arracacha, bamboo shoot, beetroot, burdock, broadleaf arrowhead, camas, canna, carrot, cassava, celeriac, chinese artichoke, daikon, earthnut pea, elephant foot yam, ensete, galangal, ginger, ginseng, hamberg parsley, lotus root, jerusalem artichoke, jicama, mashua, parsley root, parsnip, pignut, potato, prairie turnip, radish, rutabaga, salsify, scorzonera, skirret, swede, sweet potato, taro, ti, tigernut, turnip, ulluco, wasabi, water caltrop, water chestnut, yalcon, yam, avocado, bell pepper, bitter melon, bitter gourd, breadfruit, chayote, cucumber, ivy gourd, eggplant, aubergine, brinjal, luffa, nopal, okra, pepper, pumpkin, squash, sweet corn, sweet pepper, tinda, tomatillo, tomato, west indian gherkin, winter melon, zucchini, courgette, artichoke, broccoli, caper, cauliflower, courgette flower, globe artichoke, squash blossom, asparagus, prussian asparagus, bamboo shoot, celery, leek, wild leek, lemongrass, rhubarb, cardoon, kurrat, galangal, amaranth, arugula, beet green, bok choy, borage, broccoli rabe, brussel sprout, cabbage, catsear, celery, celtuce, chard, chaya, chickweed, chicory, chinese mallow, chive, choi sum, collard green, common purslane, corn salad, cress, dandelion, endive, fat hen, fiddlehead, fluted pumpkin, garden rocket, golden samphire, good king henry, greater plantain, kai lan, kale, komatsuna, kuka, lagos bologi, lamb's lettuce, lamb's quarters, land cress, leek, lettuce, lizard's tail, malabar spinach, melokhia, miner's lettuce, mizuna green, mustard green, napa cabbage, new zealand spinach, orache, pak choy, paracress, pea, poke, radicchio, samphire, sculpit, stridolo, sea beet, sea kale, sierra leoni bologi, soko, sorrel, spinach, summer purslane, swiss chard, tatsoi, turnip green, watercress, water spinach, wheatgrass, winter purslane, yarrow, yao choy, cardoon, chive, florence fennel, elephant garlic, garlic, garlic chive, kohlrabi, onion, pearl onion, potato onion, spring onion, tree onion, welsh onion, quamash, scallion, shallot, rice, barley, oat, barley, rye, corn, wheat, buckwheat, millet, sorghum, spelt, quinoa, chicory, molasses, beet root, fonio, palmer's grass, teff, triticale, cattail, chia, flax, grain amaranth, canihua, pitseed goosefoot, wattleseed, almond, cashew, chestnut, coconut, hazelnut, macadamia, peanut, pecan, pine nut, pistachio, walnut, acorn, brazil nut, gingko nut, shea nut, beechnut, candlenut, kurrajong, mongongo, palm nut, karuka, red bopple nut, hickory, jacknut, pepea nut, yellow walnut, cycad, munya nut, monkey puzzle nut, paradise nut, colocynth, filbert, mamoncillo, maya nut, ogbono nut, water caltrop, juniper, podocarp, cempedak, coffee, egusi, fox nut, jackfruit, malabar gourd, nutmeg, breadnut, betel nut, cola nut, pili nut, olive, drumstick, okra, pumpkin seed, sesame seed, sunflower seed, lotus seed, hemp seed, green bean, red bean, azuki bean, black bean, back eyed bean, white bean, yellow bean, common bean, broad bean, pinto bean, soybean, lima bean, kidney bean, pea bean, potato bean, dolichos bean, moth bean, mung bean, ricebean, runner bean, tepary bean, urad bean, velvet bean, winged bean, yardlong bean, chickpea, pigeon pea, snap pea, snow pea, lentil, lupin, tarwi, carob, guar, horse gram, cocoa bean, coffee bean, bambara groundnut, cowpea, hyacinth bean, lablab, moringa oleifera, sterculia, yam bean, alfalfa, clover, mesquite, carob, tamarind, wisteria, aonori, arame, carola, caulerpa, dabberlocks, dulse, hijiki, kombu, layer, mozuku, nori, ogonori, sea grape, sea lettuce, sea weed, wakame, truffle, mushroom, bean sprout, acai, aloe vera, arnica, ashoka, asthma weed, astragalus, barberry, belladonna, bitter leaf, bitter orange, black cohosh, blessed thistle, cat's claw, cayenne, chaparral, chasteberry, cinchona, coffee senna, comfrey, digitalis, dong quai, ephedra, european mistletoe, evening primrose, fenugreek, feverfew, flaxseed, gingko, goldenseal, hoodia, horse chestnut, horsetail, jamaica dogwood, jojoba, kava, khat, konjac, kratom, kanna, licorice root, marigold, moringa oleifera, milk thistle, neem, opium poppy, purple coneflower, passion flower, red clover, syrian rue, st john's wort, saw palmetto, tea tree oil, thunder god vine, tulasi, turmeric, umckaloabo, valerian, white willow, yerba santa, ajwain, akudjura, alexanders, alkanet, alligator pepper, angelica, anise, anise hyssop, aniseed myrtle, annatto, apple mint, artemisia, asafoetida, asarabacca, avens, avocado leaf, barberry, bay leaf, bee balm, boldo, borage, black cardamom, blue fenugreek, caraway, cardamom, cassia, cayenne pepper, celery leaf, celery seed, chervil, chicory, cicely, cilantro, clary, costmary, cubeb pepper, cudweed, culantro, curry leaf, curry plant, dill seed, epazote, fenugreek, file powder, fingerroot, galangal, galingale, ganoderma, golpar, grains of paradise, grains of selim, houttuynia cordata, huacatay, indonesian bay leaf, jimbu, kaffir lime leaves, kala zeera, kawakawa seeds, kencur, keluak, kinh gioi, kokam seed, korarima, koseret leaves, lemon myrtle, lemon ironbark, lemon verbena, leptotes bicolor, lesser calamint, lime flower, lovage, mahlab, marjoram, mastic, mountain horopito, musk mallow, nasturtium, nigella, njangsa, neem, olida, orris root, pandan flower, pandan leaf, paprika, paracress, peppermint gum leaf, perilla, pandanus amaryllifolius, quassia, ramsons, rice paddy herb, rue, saffron, salad burnet, salep, sassafras, savory, silphium, shiso, sorrel, spikenard, star anise, sumac, sweet woodruff, tarragon, voatsiperifery, water-pepper, wattleseed, wild betel, willow herb, wood avens, and woodruff.

32. The snack system of claim 27 wherein said vegetable oil flavor is selected from group consisting of corn oil, canola oil, peanut oil, olive oil, coconut oil, soybean oil, sesame oil, sunflower oil, flaxseed oil, grape seed oil, pumpkin seed oil, safflower oil, argan oil, rice bran oil, palm oil, palm kernel oil, almond oil, avocado oil, cottonseed oil, hemp oil, mustard oil, macadamia oil, groundnut oil, teaseed oil, walnut oil, cashew oil, castor oil, colza oil, hazelnut oil, linseed oil, manila oil, mongongo nut oil, mustard oil, pecan oil, perilla oil, pine nut oil, pistachio oil, poppyseed oil, rapeseed oil, jojoba oil, and watermelon seed oil.

33. The snack system of claim 27 wherein said meat flavor is selected from group consisting of chicken, duck, goose, turkey, quail, rabbit, sheep, goat, cow, pig, deer, bison, buffalo, crocodile, frog, turtle, snail, worm, bacon, sausage, animal tissue, animal organ, animal tongue, animal tail, animal feet, animal liver, animal stomach, animal intestine, animal brain, animal heart, animal skin, animal tendon, animal fat, animal bone marrow, and animal blood.

34. The snack system of claim 27 wherein said seafood flavor is selected from group consisting of squid, cuttle fish, shrimp, crab, crayfish, octopus, clam, oyster, scallop, lobster, abalone, conch, jellyfish, prawn, krill, mussel, sea cucumber, sea urchin, eel, snail, shrimp roe, crab roe, lobster roe, fish roe, and fish.

35. The snack system of claim 4 wherein said at least one nutritional ingredient comprises essential nutrient selected from group consisting of vitamin, dietary mineral, amino acid, and fatty acid; said vitamin includes vitamin A, vitamin B1, vitamin B2, vitamin B3, vitamin B5, vitamin B6, vitamin B7, vitamin B9, vitamin B12, vitamin C, vitamin D, vitamin E, and vitamin K; said dietary mineral includes calcium, phosphorus, potassium, sulfur, sodium, chlorine, magnesium, iron, cobalt, copper, zinc, molybdenum, iodine, selenium, manganese, nickle, chromium, fluorine, boron, and strontium; said amino acid includes histidine, isoleucine, leucine, lysine, methionine, cysteine, phenylalanine, tyrosine, threonine, tryptophan, and valine; said fatty acid includes alpha-linoleic acid, and linoleic acid; and said at least one nutritional ingredient is in form selected from group consisting of extract, juice, sauce, liquid, oil, gel, jelly, wax, cream, paste, powder, thin layer, small particles, and small pieces.

36. The snack system of claim 4 wherein said at least one health ingredient comprises medication and other health ingredient; said medication is mixed into said snack for ease of consumption; said other health ingredient is selected from group consisting of electrolyte, lactase, yeast, propolis, and quinine; and said at least one health ingredient is in form selected from group consisting of extract, juice, sauce, liquid, oil, gel, jelly, wax, cream, paste, powder, thin layer, small particles, and small pieces.

37. The snack system of claim 4 wherein said at least one other ingredient comprises food additive selected from group consisting of acid, acidity regulator, alcohol, anticaking agent, antifoaming agent, antioxidant, bulking agent, coagulation agent, food coloring, color retention agent, emulsifier, flavor enhancer, flour treatment agent, gelling agent, glazing agent, humectant, leavening agent, tracer gas, preservative, stabilizer, sweetener, tenderizer, and thickener.

38. The snack system of claim 1 wherein said at least one ingredient further comprises at least one binding agent which is an edible binder and is selected from group consisting of protein component and starch component; said protein component is selected from group consisting of gelatin, beaten egg, and egg white; said starch component is selected from group consisting of vegetable gum, vegetable starch, and vegetable flour; said vegetable gum is selected from group consisting of agar, alginic acid, sodium alginate, carrageenan, gum arabic, gum ghatti, gum tragacanth, karaya gum, guar gum, locust bean gum, beta glucan, chicle gum, dammar gum, glucomannan gum, mastic gum, psyllium seed husk, spruce gum, tara gum, gellan gum, and xanthan gum; said vegetable starch and vegetable flour are selected from group consisting of root vegetable, cereal grain, nut, seed, bean, pectin, sago, and tapioca; said at least one binding agent can be cooked by method selected from group consisting of mixing with water, boiling, and steaming; and said gelatin from fish and animal congeals below 15 degrees Celsius.

39. The snack system of claim 1 wherein said snack with at least one binding agent comprising gelatin can be refrigerated to serve cold but not reheated because gelatin from fish and animal congeals below 15 degrees Celsius; and said snack with at least one binding agent selected from group consisting of beaten egg, egg white, and starch component can be served at room temperature, refrigerated to serve cold, and reheated to serve hot.

40. The snack system of claim 1 wherein said snack having packaging selected from group consisting of individual packaging and multiple packaging; said packaging is in air tight container; and said packaging having material selected from group consisting of paper, plastic, metal, and glass.
